(19) United States
(12) Patent Application Publication
(10) Pub. No.: US 2009/0054763 A1
(43) Pub. Date: Feb. 26, 2009

(54) SYSTEM AND METHOD FOR SPECTROSCOPIC PHOTOACOUSTIC TOMOGRAPHY

(75) Inventors: Xueding Wang, Canton, MI (US); David Chamberland, Medford, OR (US); Paul Carson, Ann Arbor, MI (US); Brian Fowlkes, Ann Arbor, MI (US)

Correspondence Address: BROOKS KUSHMAN P.C., 1000 TOWN CENTER, TWENTY-SECOND FLOOR, SOUTHFIELD, MI, 48075, US

(73) Assignee: THE REGENTS OF THE UNIVERSITY OF MICHIGAN, Ann Arbor, MI (US)

(21) Appl. No.: 12/161,623

(22) PCT Filed: Jan. 19, 2007
(86) PCT No.: PCT/US07/60762

§ 371 Date: Jul. 21, 2008

Related U.S. Application Data
(60) Provisional application No. 60/760,178, filed on Jan. 19, 2006.
(60) Provisional application No. 60/760,175, filed on Jan. 19, 2006.

(51) Int. Cl. A61B 5/05; A61B 8/00

(52) U.S. Cl. 600/425; 600/453

(57) ABSTRACT

A system and method for spectroscopic photoacoustic tomography of a sample include at least one light source configured to deliver light pulses at two or more different wavelengths to the sample. An ultrasonic transducer is disposed adjacent to the sample for receiving photoacoustic signals generated due to optical absorption of the light pulses by the sample. A control system is provided in communication with the ultrasonic transducer for reconstructing photoacoustic tomographic images from the received photoacoustic signals, wherein upon application of light pulses of two or more different wavelengths to the sample, the control system is configured to determine the local spectroscopic absorption of substances at any location in the sample. The system may further provide for one or more of ultrasound imaging. Doppler ultrasound imaging, and diffuse optical imaging of the sample.

### CROSS-REFERENCE TO RELATED APPLICATIONS

This application claims the benefit of U.S. provisional application Ser. No. 60/760,178 filed Jan. 19, 2006 and U.S. provisional application Ser. No. 60/760,175 filed Jan. 19, 2006, both of which are incorporated by reference herein.

### BACKGROUND OF THE INVENTION

1. Field of the Invention

This invention relates to spectroscopy and photoacoustic tomography.

2. Background Art

Photoacoustic tomography (PAT) may be employed for imaging tissue structures and functional changes and describing the optical energy deposition in biological tissues with both high spatial resolution and high sensitivity. PAT employs optical signals to generate ultrasonic waves. In PAT, a short-pulsed electromagnetic source—such as a tunable pulsed laser source, pulsed radio frequency (RF) source or pulsed lamp—is used to irradiate a biological sample. The photoacoustic (ultrasonic) waves excited by thermoelastic expansion are then measured around the sample by high sensitive detection devices, such as ultrasonic transducer(s) made from piezoelectric materials and optical transducer(s) based on interferometry. Photoacoustic images are reconstructed from detected photoacoustic signals generated due to the optical absorption in the sample through a reconstruction algorithm, where the intensity of photoacoustic signals is proportional to the optical energy deposition.

Optical signals, employed in PAT to generate ultrasonic waves in biological tissues, present high electromagnetic contrast between various tissues, and also enable highly sensitive detection and monitoring of tissue abnormalities. It has been shown that optical imaging is much more sensitive to detect early stage cancers than ultrasound imaging and X-ray computed tomography. The optical signals can present the molecular conformation of biological tissues and are related to significant physiologic parameters such as tissue oxygenation and hemoglobin concentration.

Traditional optical imaging modalities suffer from low spatial resolution in imaging subsurface biological tissues due to the overwhelming scattering of light in tissues. In contrast, the spatial resolution of PAT is only diffraction-limited by the detected photoacoustic waves rather than by optical diffusion; consequently, the resolution of PAT is excellent (60 microns, adjustable with the bandwidth of detected photoacoustic signals). Besides the combination of high electromagnetic contrast and high ultrasonic resolution, the advantages of PAT also include good imaging depth, relatively low cost, non-invasive, and non-ionizing.

Photoacoustic spectroscopy (PAS) is an analytical method that involves stimulating a sample by light and subsequently detecting sound waves emanating from the sample. Typically, only a narrow range of wavelengths of light are introduced into a sample. Such narrow range of wavelengths of light can be formed by, for example, a laser. Utilization of only a narrow range of wavelengths can enable preselected molecular transitions to be selectively stimulated and studied. The subsequent non-radiative relaxation that occurs is then measured as an acoustic or ultrasonic signal by high-sensitivity ultrasonic detectors such as piezoelectric crystals, microphones, optical fiber sensors, laser interferometers or diffraction sensors. Because most biological chromophores and molecules relax primarily through non-radiative processes, PAS can be an extremely sensitive means of detection. For example, the use of photoacoustic spectroscopy for glucose testing in blood and human tissue can provide greater sensitivity than conventional spectroscopy. An excellent correlation between the photoacoustic signal and blood glucose levels has been demonstrated on index fingers of both healthy and diabetic patients.

Currently, photoacoustic spectroscopy is employed in medicine, biology and other areas primarily as a sensing technique without providing high resolution morphological information of studied samples. For example, in medical applications, photoacoustic spectroscopy has been employed to study blood glucose concentration as well as hemoglobin oxygen saturation in biological samples. However, the spatially distributed concentrations of absorbing chromophores as well as their changes as results of functional physiological activities are not presented with pin-point accuracy.

Diffuse optical tomography (DOT), including near-infrared spectroscopy (NIRS), is emerging as a viable new biomedical imaging modality. In DOT, light in the ultraviolet, visible or near-infrared (NIR) region is delivered to a biological sample. The diffusely reflected or transmitted light from the sample is measured and then used to probe the absorption and scattering properties of biological tissues. DOT is now available that allows users to obtain cross-sectional and volumetric views of various body parts. Currently, the main application sites are the brain, breast, limb, and joint.

More recently, there has been great interest in adapting the methodologies of DOT to fluorescent imaging and bioluminescence imaging. One advantage of such a method is that it presents the high contrast and specificity of fluorescent dye tagging. Although the spatial resolution is limited when compared with other imaging modalities, DOT provides access to a variety of physiological parameters that otherwise are not accessible, including sub-second imaging of hemodynamics and other fast-changing processes. Furthermore, DOT can be realized in compact, portable instrumentation that allows for bedside monitoring at relatively low cost.

Ultrasound imaging (US) involves placing a transducer against the skin of the patient near the region of interest, for example, against the back to image the kidneys. The ultrasound transducer combines functions like a stereo loudspeaker and a microphone in one device: it can transmit sound and receive sound. This transducer produces a stream of inaudible, high frequency sound waves which penetrate into the body and bounce off the organs inside. The transducer detects sound waves as they bounce off or echo back from the internal structures and contours of the organs. Different tissues reflect these sound waves differently, causing a signature which can be measured and transformed into an image. The ultrasound instrument processes the echo information and generates appropriate dots which form the image. The brightness of each dot corresponds to the echo strength, producing a gray scale image. Conventional US includes two dimensional (2D) and three dimensional (3D) ultrasound imaging employing either a 1D, 1.5D or 2D ultrasonic transducer array.

Doppler ultrasound is a form of flow imaging based on the pulse-echo technique. The Doppler effect is a change in the frequency of a wave resulting from motion of the wave source or receiver or, in the case of a reflected wave, motion of the reflector. In medicine, Doppler ultrasound is used to detect and measure blood flow, and the major reflector is the red blood cell. The Doppler shift is dependent on the insonating frequency, the velocity of moving blood, and the angle between the sound beam and the direction of moving blood.

### BRIEF DESCRIPTION OF THE DRAWINGS

FIG. 1 is a schematic diagram of a SPAT system according to the present invention;

FIG. 2 depicts a circular transducer array which can be applied in SPAT according to the present invention; and

FIG. 3 is a schematic diagram of a multi-modality imaging system according to one aspect of the present invention including photoacoustic tomography, ultrasound imaging, and diffuse optical imaging.

### DETAILED DESCRIPTION OF THE INVENTION

As required, detailed embodiments of the present invention are disclosed herein; however, it is to be understood that the disclosed embodiments are merely exemplary of the invention that may be embodied in various and alternative forms. The figures are not necessarily to scale, some features may be exaggerated or minimized to show details of particular components. Therefore, specific structural and functional details disclosed herein are not to be interpreted as limiting, but merely as a representative basis for teaching one skilled in the art to variously employ the present invention.

The present invention includes a system and method for spectroscopic photoacoustic tomography (SPAT) which may yield high resolution images and point-by-point spectral curves for substance identification within a three-dimensional specimen, such as biological organs. In medical diagnostic imaging and therapeutic monitoring, the system and method according to the present invention are able to achieve a microscopic view into specimens and may provide not only morphological information, but also functional molecular and biochemical information of tissues.

The SPAT system and method according the present invention may provide a high resolution three dimensional map of a specimen while simultaneously being able to provide spectral curves on a point-by-point basis in a volumetric fashion of the same specimen. The point-by-point spectroscopic information is able to manifest the presence, concentrations, and changes of the biological and biochemical substances in the localized areas in the specimen with both high sensitivity and high specificity.

In the SPAT system and method according to the present invention, a light source with short pulse duration (e.g., on the order of nanoseconds) and narrow linewidth (e.g., on the order of nanometers) may be used to irradiate a sample under study. The wavelength of the light may be tunable over a broad region (for example, but not limited to, from 300 nm to 1850 nm). By varying the light wavelength in the tunable region and applying laser pulses at two or more wavelengths to the biological sample sequentially, high resolution photoacoustic images of the sample at each wavelength can be obtained. Additionally, with the measured photoacoustic images as a function of wavelength, the local spectroscopic absorption of each point in the sample can be studied, which presents both morphological and functional information. At each voxel in a three dimensional area, a spectroscopic curve indicating the concentration of various absorbing materials can be produced. The SPAT system and method according to the present invention therefore allows for the study of spectroscopic absorption properties in biological tissues with high sensitivity, high specificity, good spatial resolution and good imaging depth.

In medical imaging and diagnosis, a biological specimen can be imaged with SPAT in accordance with the present invention in three dimensions, and also produce spectroscopic curves at each point within the three dimensional specimen. The point-by-point spectroscopic curves enable the spectral identification and mapping of any substance with a unique spectral curve including exogenously added substances, such as molecular or cellular probes, markers, antibodies, contrast agents, and the like, and endogenous biological and biochemical substances in localized areas in the specimen including, but not limited to, glucose, hemoglobin, lipid, water, and cytochromes. The spatially and volumetrically distributed spectroscopic information can be used for noninvasive serial in vivo identification of different intrinsic biological tissues and extrinsic substances for both diagnostic and therapeutic purposes, such as in the setting of disease diagnosis, disease progression, and monitoring of tissue changes during treatments not limited to drug therapies.

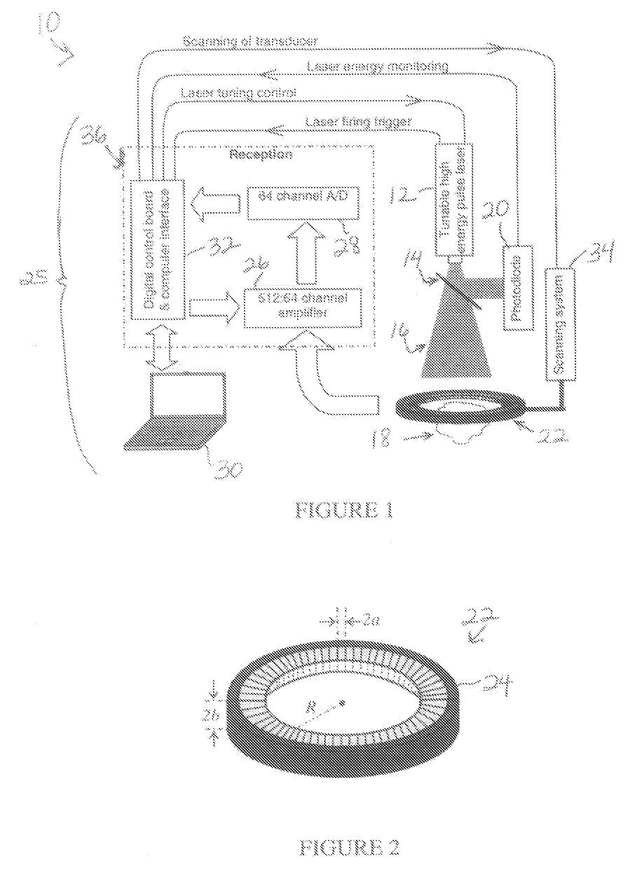

The SPAT system according to the present invention includes (a) laser delivery and wavelength tuning, (b) photoacoustic signal generation and reception, © reconstruction and display of the photoacoustic tomographic image, and (d) generation and analysis of point-by-point spectroscopic information. FIG. 1 depicts a schematic diagram of a SPAT system according to the present invention, indicated generally by reference numeral 10. According to one aspect of the present invention, at least one light source or laser 12, such as an optical parametric oscillator (OPO) laser system pumped by an Nd:YAG laser working at 532 nm (second-harmonic), may be used to provide pulses (e.g., ˜5 ns) with a tunable wavelength, such as ranging between 680 nm and 950 nm. Other spectrum regions can also be realized by choosing other tunable laser and systems or lamps, e.g. dye laser, Ti:Sapphire laser and OPO laser pumped by 355 nm light (Nd:YAG at third-harmonic). Of course, other configurations are also fully contemplated. The selection of the laser spectrum region depends on the imaging purpose, specifically the biochemical substances to be studied. Through free space or an optical fiber bundle, laser light 16 may be delivered to the sample 18 with an input energy density below the ANSI safety limit. The delivered laser energy can be monitored by an optical sensor (e.g. photodiode) 20, which may be facilitated by beam splitter 14.

Instead of tuning the wavelength of one laser source, such as laser 12, to realize spectroscopic measurement, two or more lasers each operating at a different wavelength may also be employed for SPAT according to the present invention. In this case, the time used for wavelength tuning can be saved, and hence high speed SPAT can be achieved.

The spatially-distributed optical energy in the sample 18 generates proportionate photoacoustic waves due to the optical absorption of biological tissues (i.e., optical energy deposition), which may be coupled into a transducer 22, such as a high-sensitivity wide-bandwidth ultrasonic transducer. Water, oil, ultrasonic coupling gel, or the like can be used as the coupling material between the sample 18 and transducer 22. Other high sensitive ultrasound detection devices, such as an optical transducer based on interferometry, can be used instead of ultrasonic transducer 22.

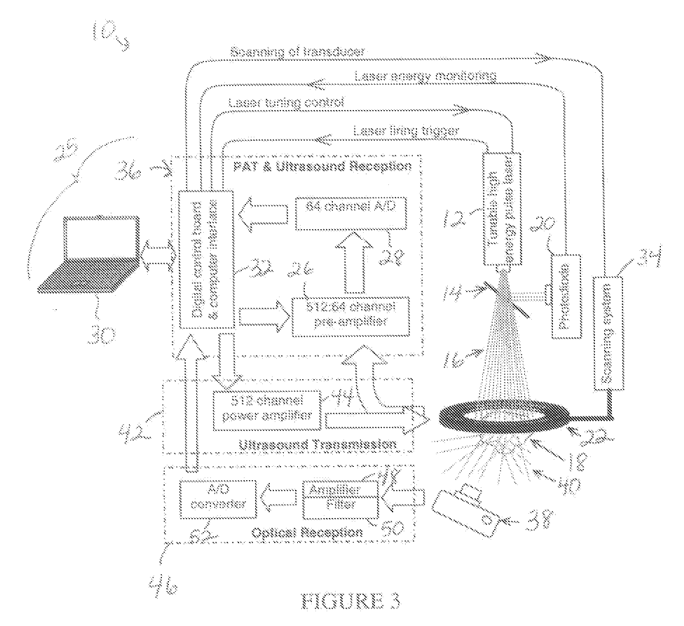

The detailed geometry of a circular transducer array 24 which may be used with the SPAT system according to the present invention is shown in FIG. 2. Array 24 is a 1D array that is able to achieve 2D imaging of the cross section in the sample 18 surrounded by the array 24 with single laser pulse. The imaging of a 3D volume in the sample 18 can be realized by scanning the array 24 along its axis. In order to achieve 3D photoacoustic imaging at one wavelength with a single laser pulse, a 2D transducer array could instead be employed for signal detection.

The parameters of ultrasonic transducer 22 include element shape, element number, array geometry, array central frequency, detection bandwidth, sensitivity, and others. The design of transducer 22 in the SPAT system according to the present invention may be determined by the shape of the studied sample 18, the expected spatial resolution and sensitivity, the imaging depth, and others. For example, for SPAT of human finger or toe joints with inflammatory arthritis, a circular array 24 can be applied as in FIG. 2. According to one aspect of the present invention, the design of array 24 may be: central frequency of 7.5 MHZ, bandwidth of 80%, pitch size 2a of 0.3 mm, array size of 50 mm in diameter, number of element of 512, and array elevation height 2b of 0.2 mm. This transducer 22 may realize imaging resolution at 200 micrometers in human finger or toe joints. Of course, other configurations of transducer 22 and array 24 are also fully contemplated.

With reference again to FIG. 1, the photoacoustic signals detected by transducer 22 may be communicated to a control system 25, which includes a processor, such as a computer 30, and reception circuitry 36. Reception circuitry 36 may include an amplifier 26 (e.g., 64 channel), an A/D converter 28 (e.g., 64 channel), and an a digital control board and computer interface 32. Digital control board and computer interface 32 may also receive the triggers from laser 12 and record the laser pulse energy detected by photodiode 20. At the same time, computer 30 may also control the tuning of the wavelength of laser 12 through digital control board and computer interface 32. Still further, the scanning of transducer 22 to detect photoacoustic signals may be accomplished through a scanning system 34 and digital control board and computer interface 32. Photoacoustic tomographic images may be reconstructed from detected signals through a reconstruction algorithm. After one photoacoustic image has been obtained, computer 30 may record the data and tune laser 12 to the next wavelength. It is understood that control system 25 shown in FIG. 1 is only an example, and that other systems with similar functions may also be employed in the SPAT system 10 according to the present invention for control and signal receiving.

One advantage of the spectroscopic photoacoustic tomography (SPAT) system according to the present invention is that spectroscopic information can be obtained on a point-by-point basis in a three-dimensional sample 18. This enables the study of a sample presenting both morphological information and spectroscopic information with both high spatial resolution and high sensitivity. Comparing to photoacoustic tomography (PAT) that can present biological tissue properties and changes in a three dimensional space, spectroscopic photoacoustic tomography according to the present invention provides extra spectroscopic information that is sensitive to important functional and biochemical properties in tissues at molecular and cellular levels. Therefore, unlike PAT and PAS, the SPAT system and method of the present invention provide three-dimensional imaging with additional point-by-point spectral identification to obtain a more comprehensive description of a sample.

Other advantages of the present invention may include the use of non-ionizing radiation non-invasively, wherein both the optical energy and ultrasonic energy used have low power and pose no known hazards to animals or humans. The system and method of the present invention provide a combination of high spectroscopic optical contrast and high ultrasonic resolution, and provide a functional imaging ability which is sensitive not only to different soft tissues that have different optical properties, but also to functional changes in biological tissues. The SPAT system and method also provide a molecular and cellular imaging ability, where spectroscopic information manifests the presence, concentrations and changes of the biological and biochemical substances in the localized areas in the specimen with both high sensitivity and high specificity. The system and method of the present invention also provide good penetration on the order of multiple centimeters into biological tissues when the spectrum in the near-infrared and infrared regions is studied. Furthermore, no speckle effect is present, as photoacoustic waves travel one way to reach the ultrasonic transducer array 24 rather than two ways as in a conventional pulse-echo imaging mode. This minimizes the speckle effect caused by multiple scattering, which is a key issue in conventional pulse-echo ultrasonography.

In accordance with the present invention, the object to be studied using the SPAT system and method can be any sample, such as a living organism, animals, or humans. The spectroscopic images of the sample 18 may be generated invasively or non-invasively, that is, while the skin and other tissues covering the organism are intact. The SPAT system and method according to the present invention could also be used in industrial settings for any medium which is favorable to optical signal-produced thermoelastic expansion causing acoustic wave propagation including, but not limited to, liquid chemical purity measurements. In accordance with the present invention, the SPAT system and method could be customized to a particular type of tissue or material as a scan utilizing the spectrum of light (multiple wavelengths) that most characterizes this type of tissue or material.

Transducer 22 can be any proper ultrasound detection device, e.g. single element transducers, 1D or 2D transducer arrays, optical transducers and transducers of commercial ultrasound machines, and others. The photoacoustic signals can be scanned along any surfaces around the sample. Moreover, detection at the detection points may occur at any suitable time relative to each other. The signal between the sample 18 and transducer 22 may be coupled with any transparent ultrasound coupling material, such as water, mineral oil, ultrasound coupling gel, or other suitable substance.

The light source 12 according to the present invention may be any device that can provide short light pulses with high energy, short linewidth and tunable wavelength, such as, but not limited to, a Ti:Sapphire laser, OPO systems, dye lasers and arc lamps. The wavelength spectrum of the light pulses may be selected according to the imaging purpose, specifically absorbing substances in the sample 18 to be studied. The studied spectral region may range from ultraviolet to infrared (300 nm to 1850 nm), but is not limited to any specific range. The light energy may be delivered to the sample 18 through any methods, such as free space beam path and optical fiber(s). The intensity of the light pulses may be monitored with any sensor 20, such as photodiode and PMT.

According to the present invention, the reconstruction used in the SPAT system and method to generate photoacoustic signals can be any basic or advanced algorithms, such as simple back-projection, filtered back-projection and other modified back-projection methods. The reconstruction of photoacoustic tomographic images may be performed in both spatial domain and frequency domain. Before or after reconstruction, any signal processing methods can be applied to improve the imaging quality. Images may be displayed on computer 30 or another display.

Computer 30 may control light source 12, may control and record the photoacoustic signal data, may reconstruct photoacoustic images, and may generate and analyze point-by-point spectroscopic information. A “computer” may refer to any suitable device operable to execute instructions and manipulate data, for example, a personal computer, work station, network computer, personal digital assistant, one or more microprocessors within these or other devices, or any other suitable processing device.

The SPAT system and method according to the present invention can be performed based on both intrinsic and extrinsic contrasts. System 10 may be used to study the intrinsic optical properties in the sample 18 without applying contrast agents. Furthermore, system 10 may be used to image a sample 18 in three dimensions and also enable the generation of spectroscopic curves of extrinsic substances added to biological tissues. Added extrinsic substances include, but are not limited to, those substances which may enhance an image or localize within a particular region, or any type of therapy including pharmaceutical applications. Possible employed contrast agents include quantum dots, dyes, nano-particles, absorbing proteins, and other absorbing substances.

The reception of photoacoustic signals can be realized with any proper designs of control system 25. Circuitry 36 performs as an interface between computer 30 and transducer 22, laser 12, and other devices. “Interface” may refer to any suitable structure of a device operable to receive signal input, send control output, perform suitable processing of the input or output or both, or any combination of the preceding, and may comprise one or more ports, conversion software, or both. A component of a reception system may comprise any suitable interface, logic, processor, memory, or any combination of the preceding.

The SPAT system and method according to the present invention could also be used for point to point treatment, i.e. once a characteristic spectral curve is detected at any three-dimensional location within the sample, thermal or photo or acoustic signals could be directed to that location for therapies needing thermal ablation or photoactivation of a pharmaceutical compound.

In accordance with the present invention, the SPAT system and method may further include other imaging modalities, such as diffuse optical imaging and ultrasound imaging technologies, and can yield photoacoustic, functional spectroscopic photoacoustic, diffuse optical, 2D or 3D ultrasound, and Doppler ultrasound diagnostic information. With reference to FIG. 3, system 10 according to the present invention includes an ultrasonic transducer 22, a light source 12, and an optical detector 38. Pulsed light from light source 12 can induce photoacoustic signals in an imaged sample 18 that are detected by ultrasonic transducer 22 to generate 2D or 3D photoacoustic tomographic images of the sample 18. By tuning the wavelength of the light, functional spectroscopic photoacoustic tomography of the sample 18 can also be realized. At the same time, the light 40 scattered upon delivery to the sample 18 can be measured in either forward mode (transmittance) or backward mode (diffuse reflectance) by optical detector 38 to achieve diffuse optical imaging of the sample 18. When multiple wavelengths in the NIR region are applied, NIRS of the sample 18 is achievable. Ultrasonic transducer 22 may also be used to realize conventional gray scale ultrasound imaging and Doppler ultrasound of the sample 18 by using ultrasonic transducer 22 as both a transmitter and receiver of ultrasound signals and appropriate existing signal processing circuitry 36.

Therefore, multi-modality system 10 according to the present invention can generate photoacoustic images, optical images, and ultrasound images of the same sample 18 at the same time. The photoacoustic image presents the optical absorption distribution in biological tissues, while spectroscopic photoacoustic data reveal not only the morphological information but also functional biochemical information in biological tissues. Photoacoustic images have both high optical contrast and high ultrasonic spatial resolution. Optical images include both scattering images and absorption images of the sample 18. Although the spatial resolution of optical images is limited compared with the photoacoustic results, optical imaging is able to access both the absorption and scattering properties of the sample 18 at the same time with very high sensitivity and specificity. Besides the scattering properties, optical imaging can also probe the intensities of fluorescent signals that cannot be studied by photoacoustic technology. In comparison with photoacoustic images and optical images that are all based on the optical contrast, ultrasound images of the sample 18 present the mechanical contrast in biological tissues and probe the tissue acoustic properties, including density, acoustic velocity, elasticity, speed of flow, etc. The spatial resolution of ultrasound images is similar to that of photoacoustic images and higher than that of optical images. According to the present invention, the photoacoustic, optical and ultrasound imaging results of the same sample 18 may be combined together through image registration and used to provide very comprehensive diagnostic information.

Therefore, system 10 may include transmission and receiving of ultrasound signals and generation of ultrasound images, and detection of transmitted or diffusely reflected optical signals and reconstruction of optical images. For ultrasound imaging, ultrasonic transducer 22 can perform both ultrasound signal transmission and receiving. Alternatively, an additional ultrasonic transducer could be used for ultrasound imaging. Reception circuitry 36 may also be employed for ultrasound signal receiving and processing, where the ultrasound signal transmission may be achieved through an ultrasound transmission system 42 controlled by digital control board and computer interface 32. Ultrasound transmission system 42 is capable of generating high voltage pulses and corresponding delays for each element of transducer 22, and may include an amplifier 44 (e.g., 512 channel power amplifier). A conventional pulse-echo technique may be used for the pure ultrasound imaging.

The whole array 24 or overlapping subarrays can be used to transmit and receive ultrasound pulses and then generate ultrasound images of the sample 18 through the technique of synthetic aperture. Multiple transmissions can be used for each subarray position in order to create multiple focal zones and thereby achieve uniform illumination along the propagation path. System 10 according to the present invention can realize not only gray scale ultrasound images to present tissue morphology in 2D or 3D space, but also Doppler ultrasound images to depict blood flow in biological tissues.

Diffuse optical tomography of the sample 18 can be realized at the same time when photoacoustic tomography is conducted. As described above, light pulses 16 are delivered to the sample 18 to generate photoacoustic signals that are detected by ultrasonic transducer 22. At the same time, the light delivered to the sample 18 propagates in the biological tissues. The trajectories of light photons are changed quickly due to the overwhelming scattering property of tissues. The scattered photons, except those absorbed by tissues, exit the sample 18 through all the directions. Those transmitted or diffusely reflected light photons 40 may be measured out of the sample 18 and generate the distributions of optical properties, including both scattering and absorption, and concentration of fluorescent or bioluminescent sources in biological tissues. An additional light source other than laser 12 may also be used to deliver light to sample 18 for diffuse optical tomography.

In the system and method according to the present invention, the transmitted or backscattered photons may be detected by any optical sensor 38 including, but not limited to, a CCD camera, photodiode, avalanche photodiode (APD), photo-multiplier tube (PMT), or any other light detection device. The measurement of light signal can be realized through free space or optical fibers. The received optical signals containing phase, intensity, and spatial information may be sent to an optical reception system 46. Optical reception system 46 may include an amplifier 48, filter 50, and A/D converter 52 as well as other signal processing devices. The processed signals can be collected by computer 30 to generate optical images. The reconstruction of optical images, including both absorption and scattering images, can be realized through an algorithm based on diffusion theory.

The transmission and reception of ultrasound signals, and the reception of optical signals can be realized with any proper designs of circuitry and any scanning geometry. Circuitry 36, 42, 46 performs as an interface between computer 30 and transducer 22, laser 12, light detector 38, and other devices. “Interface” may refer to any suitable structure of a device operable to receive signal input, send control output, perform suitable processing of the input or output or both, or any combination of the preceding, and may comprise one or more ports, conversion software, or both. A component of a reception system may comprise any suitable interface, logic, processor, memory, or any combination of the preceding.

When fluorescent contrast agents are employed in biological tissues to enhance the imaging contrast, the incident light is divided into three parts, including: (1) photons absorbed by tissues and the fluorescent contrast agent that are transferred into heat, (2) photons absorbed by the fluorescent contrast agent that are converted into fluorescence light with different wavelength, and (3) photons transmitted or backscattered from the sample. The photons of part (1) can be measured by photoacoustic tomography, where the resulting photoacoustic images present both the intrinsic optical absorption distribution in tissues and the distribution of extrinsic contrast agent. The photons of parts (2) and (3) can be measured by diffuse optical imaging. The measurement of the photons of part (2) leads to images of absorption and scattering properties in biological tissues, and the measurement of the photons of part (3) leads to a fluorescent image.

The multi-modality system and method according to the present invention can extract complementary information of biological tissues. Photoacoustic tomography presents high resolution optical absorption information, diffuse optical imaging presents both absorption and scattering information, and ultrasound imaging presents high resolution tissue acoustic properties. All these tissue information sources may enable very comprehensive diagnosis of diseases. For example, simultaneous imaging of cancer's optical and acoustic contrasts has three major advantages. First, the images of both optical and acoustic contrasts provide more diverse and complementary information for cancer detection and diagnosis. Second, the ultrasound images are helpful for radiologists, who are already familiar with ultrasound, to extract information from photoacoustic and optical images and correlate the extracted information with the ultrasound findings. Third, the information extracted from each modality in system 10 can benefit other imaging modalities.

More particularly, the system and method according to the present invention can extract complementary information of biological tissues that cannot be realized by current existing imaging modalities. First, system 10 may describe tissue structures and properties based on both optical and acoustic contrast that may provide more diverse and complementary information for detection and diagnosis of cancers and other disorders. In the system of the present invention, findings extracted from each imaging modality can be combined together through image registration techniques. Optical contrast presents the physiology and biochemical properties of biological tissues at molecular and cellular levels, which may be added in traditional ultrasound images to help radiologists to achieve a more comprehensive diagnosis. For example, system 10 can realize very comprehensive imaging and detection of hemodynamic changes in living objects, including blood flow (by ultrasound Doppler imaging) and hemoglobin concentration and oxygenation (by PAT, SPAT, and DOT), with both high spatial and temporal resolution as well as high sensitivity and specificity.

As stated above, the information extracted from each modality in the multi-modality system of the present invention can benefit other imaging modalities. The acoustic information extracted from ultrasound imaging (e.g., acoustic heterogeneity that might cause the distortion of ultrasound signals) and the optical information extracted from diffuse optical tomography (e.g., the optical scattering of tissues that might change the distribution of optical energy) can greatly improve the imaging quality and accuracy in structural and functional photoacoustic imaging. On the other hand, the tissue morphological information extracted from photoacoustic tomography and ultrasound imaging can also improve the quality and accuracy in diffusion optical imaging. With the priori tissue anatomical information provided by PAT and/or ultrasound, local optical properties and functional parameters in biological samples can potentially be quantified with much improved specificity. With this technology, quantitative and three-dimensional imaging of fluorescent and bioluminescent sources in high scattering biological samples can also be achieved with much better accuracy and higher spatial resolution.

With the system described herein, different segments in system 10 can be most efficiently utilized. For example, laser 12 can perform as the light source for both PAT and DOT, ultrasonic transducer 22 can perform as the receiver in PAT and the transmitter and receiver in ultrasound imaging, and the PAT and ultrasound may also share one reception circuitry 36. Furthermore, the imaging of a sample 18 by one integrated multi-modality system can not only save the time and money for image acquisition in comparison with performing several imaging modalities separately, but also make image registration convenient. For example, in comparison with imaging an object in a PAT system and DOT system separately, PAT and DOT can be conducted simultaneously with the system and method according to the present invention to save time and reduce light exposure. Performing ultrasound imaging and PAT with the same transducer 22 at the same detection position makes the registration of ultrasound images and photoacoustic images of the same sample easier.

In accordance with the present invention, the reconstruction used to generate optical images can be any basic or advanced algorithms based on diffusing theory or other theories. The reconstruction of optical images may be performed in both the spatial domain and frequency domain. The ultrasound imaging may be based on pulse-echo mode, and the generation of ultrasound images may be based on synthetic aperture or any other ultrasound techniques. Before or after the generation of photoacoustic, optical and ultrasound images, any signal processing methods can be applied to improve the imaging quality.

The system and method according to the present invention could be applied to any part of the human body and adaptations could be made where a small “hand-held” transducer could be connected via cabling to a central machine housing the major components of the multi-modality system for ease of use. Also, this technology could be incorporated into invasive probes such as those used for endoscopy including, but not limited to, colonoscopy, esophogastroduodenoscopy, bronchoscopy, laryngoscopy, and laparoscopy. This system can also be used in other biomedical imaging, including those conducted on animals. The performance of this system may be invasive or non-invasive, that is, while the skin and other tissues covering the organism are intact.

Other uses of the system and method according to the present invention include industrial purposes where identification of a substance based on its spectral properties along with flow characteristics are important. Specific possibilities include material transport such as that which occurs in the oil industry during oil drilling and product transfer. Also, variables such as product purity during the refining process may be characterized. The multi-modality system of the present invention may be an improvement on existing devices used for gas analysis, i.e. commercially available gas spectrophones.

The system and method according to the present invention utilize the features of each imaging modality, many of which are complimentary and obviate the need for independent fully functioning systems, to create an enhanced hybrid image including, but not limited to, detailing the structural image of the sample, its makeup including transient characteristics such as hemoglobin content and oxygen saturation, along with blood flowing through the sample. Existing data reconstruction algorithms along with other techniques to optimize the available data may be utilized.

The combination of multiple imaging modalities in one system as described herein enables comprehensive imaging functions and features that cannot be realized by existing imaging modalities. Second, this combination is not a simple group of multiple imaging systems, but instead a systematic integration of them. The imaging modalities realized by the system according to the present invention can benefit from each other, and the different segments in this system can be most efficiently utilized. Moreover, the imaging of an object by one integrated multi-modality system can not only save the time and money for data acquisition in comparison with performing several modalities separately, but also make data registration more convenient and location more reproducible as all data is acquired in real time.

Although the system according to the present invention is described herein as including each of the photoacoustic, optical, and ultrasound imaging modalities, it is understood that system 10 may include only SPAT, may include a combination of photoacoustic tomography and ultrasound imaging, may include a combination of photoacoustic tomography and diffuse optical tomography, or any other multi-modality combination.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A system for spectroscopic photoacoustic tomography of a sample, the system comprising:
at least one light source configured to deliver light pulses at two or more different wavelengths to the sample;
an ultrasonic transducer disposed adjacent to the sample for receiving photoacoustic signals generated due to optical absorption of the light pulses by the sample; and
a control system in communication with the ultrasonic transducer for reconstructing photoacoustic tomographic images from the received photoacoustic signals wherein, upon application of light pulses of two or more different wavelengths to the sample, the control system is configured to determine the local spectroscopic absorption of substances at any location in the sample.

2. The system according to claim 1, wherein the at least one light source includes a laser having a short pulse duration.

3. The system according to claim 1, wherein the at least one light source has a tunable wavelength.

4. The system according to claim 1, wherein the at least one light source includes two or more lasers each operating at a different wavelength.

5. The system according to claim 1, further comprising an optical sensor in communication with the reception system for monitoring an energy of the delivered light pulses.

6. The system according to claim 1, wherein the control system receives a firing trigger from the light source.

7. The system according to claim 1, wherein the control system controls tuning the wavelength of the light source.

8. The system according claim 1, wherein the ultrasonic transducer includes a circular array.

9. The system according to claim 1, wherein the ultrasonic transducer is configured to transmit ultrasound signals to the sample for generating at least one of ultrasound images and Doppler ultrasound images.

10. The system according to claim 1, further comprising an additional ultrasonic transducer configured to transmit ultrasound signals to the sample for generating at least one of ultrasound images and Doppler ultrasound images.

11. The system according to claim 1, further comprising an optical detector adjacent to the sample for detecting light scattered upon delivery of the light pulses to the sample, wherein the optical detector is in communication with the control system for providing diffuse optical imaging of the sample.

12. The system according claim 11, further comprising an additional light source for delivering light to the sample for diffuse optical imaging.

13. The system according to claim 1, wherein the control system is configured to combine images of the sample through image registration.

14. The system according to claim 1, wherein the substances include intrinsic or extrinsic substances.

15. A method for spectroscopic photoacoustic tomography of a sample, comprising;
providing at least one light source;
delivering light pulses at two or more different wavelengths to the sample;
receiving photoacoustic signals generated due to optical absorption of the light pulses by the sample with an ultrasonic transducer;
reconstructing photoacoustic tomographic images from the received photoacoustic signals; and
determining the local spectroscopic absorption of substances at any location in the sample.

16. The method according to claim 15, further comprising tuning the wavelength of the at least one light source.

17. The method according to claim 15, wherein providing at least one light source includes providing two or more lasers each operating at a different wavelength.

18. The method according to claim 15, further comprising monitoring an energy of the delivered light pulses using an optical sensor.

19. The method according to claim 15, further comprising receiving a firing trigger from the light source.

20. The method according to claim 15, further comprising transmitting ultrasound signals to the sample for generating at least one of ultrasound images and Doppler ultrasound images.

21. The method according to claim 15, further comprising detecting light scattered upon delivery of the light pulses to the sample using an optical detector for providing diffuse optical imaging of the sample.

22. The method according to claim 15, further comprising combining images of the sample through image registration.

23. The method according to claim 15, further comprising directing therapeutic signals to the location within the sample.

24. A multi-modality imaging system, comprising:
a light source having a tunable wavelength, the light source configured to deliver light pulses at two or more different wavelengths to a sample;
an ultrasonic transducer disposed adjacent to the sample for receiving photoacoustic signals generated due to optical absorption of the light pulses by the sample and for transmitting ultrasound signals to the sample;
an optical detector adjacent to the sample for detecting light scattered upon delivery of the light pulses to the sample; and
a control system in communication with the ultrasonic transducer for reconstructing photoacoustic tomographic images from the received photoacoustic signals and for generating at least one of ultrasound images and Doppler ultrasound images, and in communication with the optical detector for providing diffuse optical imaging of the sample, wherein upon application of light pulses of two or more different wavelengths to the sample, the control system is configured to determine the local spectroscopic absorption of substances at any location in the sample.
